(19) United States
(12) Patent Application Publication
(10) Pub. No.: US 2004/0076662 A1
(43) Pub. Date: Apr. 22, 2004

(54) Dressing material in addition to treatment solution for use with said dressing material

(76) Inventor: Thomas Riesinger, Nurnberg (DE)

Correspondence Address: HOFFMAN WASSON & GITLER, P.C, CRYSTAL CENTER 2, SUITE 522, 2461 SOUTH CLARK STREET, ARLINGTON, VA, 22202-3843, US

(21) Appl. No.: 10/250,673

(22) PCT Filed: Jan. 9, 2002
(86) PCT No.: PCT/DE02/00032

(30) Foreign Application Priority Data
Jan. 18, 2001 (DE) 201 03 576.6
Mar. 5, 2001 (DE) 201 03 716.5
Aug. 10, 2001 (DE) 101 39 399.7
Aug. 10, 2001 (DE) 101 39 401.2

(51) Int. Cl. A61L 15/00

(52) U.S. Cl. 424/445

(57) ABSTRACT

The invention relates to a dressing material made from a plurality of layers which are adjacent to each other and joined together, namely at least one first perforated layer forming a covering on the wound, a second layer which is preferably multi-layered, soft and simultaneously absorbent in addition to a third layer forming in the outer surface of the dressing material facing away from the wound when applied. At least one chamber for distributing a liquid treatment medium is provided in the second layer, inside the series formed by said layers.

### BACKGROUND OF THE INVENTION

This invention refers to a dressing material as well to a treatment solution to be used with the dressing material.

It is basically known that the healing of wounds can be improved by providing a wet milieu, especially by using a medium which assists the healing of wounds, or at least includes a medium having a medicament, for example applying a solution or a gel. A method used in practice nowadays is that the dressing material, or a layer of this material, made of an absorbent material is soaked with the corresponding solution and then, the dressing material is applied onto the wound. With this procedure, the way of handling the dressing material is complicated and especially, when taking care of larger, or deeper wounds. Changing of the dressing material is required more often and educated or trained personal will be required to change the dressing.

It is an object of this invention to propose a dressing material which together with a simplified application results in improving the healing process.

### SUMMARY OF THE INVENTION

For solving this object a dressing material is formed of a plurality of layers with a liquid treatment medium contained therein. A treatment medium which can be used together with the dressing material, is prepared as an aqueous solution with essential components having a pH ranging from 2.5 to 3.5.

The dressing material according to the subject invention has the advantage that, in addition to its simple way of use, allows the caring of wounds in a wet milieu by using a medium, especially solutions improving the healing of wounds, and, in addition thereto that the time period within which the dressing material is to be changed, is considerably increased. For example, when using the dressing material according to the invention, it is possible to change the dressing only three days after the first treatment of the wound, which means at a time within which also a deep wound already is closed to such an extent that the danger of infections is considerably reduced. In view of the long time period the dressing material can be maintained applied to the wound, this allows to keep the wound at rest, which is necessary for fast healing, especially within the first few days subsequent to treating the wound. This is possible because the chamber for the treatment medium is provided with at least one connector extending from the dressing material for refilling and/or renewing the treatment medium.

With a preferred embodiment of the invention characterised by a very effective and low-cost structure of the dressing material, the chamber is formed by at least one channel which is divided into several sections and/or windings across the surface of the treatment material. This channel preferably is at least one piece of hose.

### BRIEF DESCRIPTION OF THE DRAWINGS

In the following, the invention is described by embodiments together with the figures of the drawings which show:

FIG. 1 in a schematic partial representation of the structure of layers of a dressing material according to the invention, whereby the individual layers which are actually connected within the dressing material in view of better understanding are shown separate from each other, in a type of explosion view;

FIG. 2 in a simplified schematic representation a top view on the material of FIG. 1;

FIG. 3 in a schematic partial representation the structure of layers of another embodiment of the dressing material according to the invention; and

FIGS. 4-7 different graphics showing the time slope of wound healings of experiments of animals.

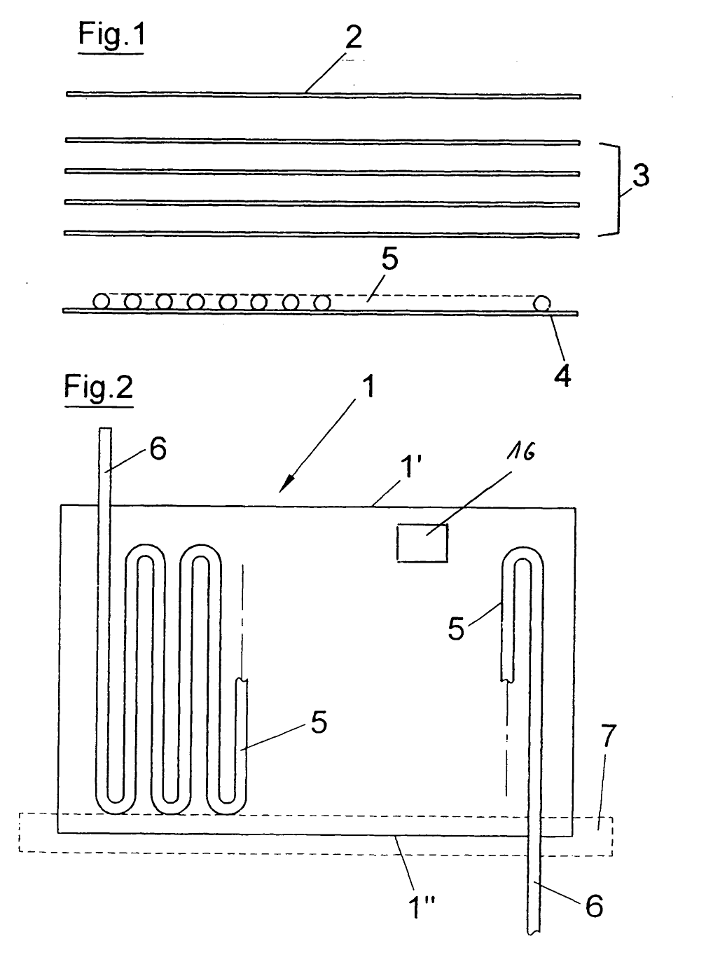

### DETAILED DESCRIPTION OF THE INVENTION

In the drawings, the dressing material 1, which preferably is suitable for taking care of chronic wounds, however, also for taking care of open, deep wounds, is substantially shown as a multi-layer flat material, for example, in the shape of a square or a rectangular cutting.

The shown embodiment of the dressing material 1 is made of a first, outer sterile layer 2 forming the coating to be applied onto the wound, and a perforated flat material, such as a perforated film or grid made of suitable plastics material, for example of PVC. The one-sheet outer layer 2 is followed by a layer 3 which is formed by several sheets of a sterile, absorbent air as well moisture permeable material, such as a coarse tissue or a non-woven material. With the embodiment according to this figure, layer 3 is made of several sheets of a sterile gauze, for example, is made of four sheets. The surface of the other side or the outer side of the dressing material 1, which when actually used is the outer surface of the dressing material opposite to the wound, is formed by a layer 4, which is made from one sheet in the shown embodiment made of a flat material or of a film, preferably a film of plastics material. The layer 4 has semipermeable characteristics which means it is permeable for air and gas, however, is not permeable for liquid. At the inner side of the layer 4 facing the layer 3 a lumen or hose portion 5 is fastened which extends meander-type nearly across the entire surface of the layer 4 and thus, across the entire surface of the dressing material 1 and forms two ends or connectors 6 which are laterally extending from the dressing material 1, with the shown embodiment in such a manner that these ends 6 are extending away from each other at two opposing lateral sides 1′ resp. 1″ of the dressing material.

The hose portion 5, which forms a channel for receiving and distributing a treatment medium, especially a liquid or aqueous treatment medium, is designed along its length extending within the treatment material 1, which means between layers 3 and 4, in such a manner that a finely divided discharge of the treatment medium within the hose portion 5 into the layer 3 will be possible. For this purpose the hose portion 5 is finely perforated. The protruding ends 6 preferably are formed by a hose length each which is without such perforations.

The individual layers 2, 3 and 4 with the hose portion 5 provided between layers 3 and 4 are connected with each other in a suitable manner, for example, along the edge of the dressing material 1 and/or dotlike also within the dressing material 1.

In use, the dressing material 1 with layer 2 is applied onto the wound to be cared of and then is fastened along the edges by means of an adhesive tape 7 or in any other manner. In order to obtain improved healing of the wound in a wet milieu, a solution suitable for wound treatment is supplied via end 6, for example, through the upper end in FIG. 2, over a time period within which this solution is discharged at the other end or connector 6, and thus the entire chamber formed by the hose portion 5 is filled with said treatment solution. Subsequent thereto both ends 6 are closed, for example, by means of a suitable plug. The treatment solution leaving the hose portion 5 is finely divided across the entire surface of the dressing material and accordingly, also across the entire wound area. The solution within the hose portion 5 is renewed and substituted several times a day, i.e. two times a day.

By means of the dressing material, according to the invention, the wound is kept occlusive and wet in an optimum manner so that a substantially improved healing result for the wound will be obtained. The special advantage of the dressing material, according to the invention, is to be seen in that changing the dressing only will be required after several days, for example, every three days only so that also the first change of the dressing material generally is required after a time period, within which even a deep open wound has improved to such an extent that the danger of an additional infection by changing the dressing can be basically excluded. In view of the long time interval between applying a dressing material and the first change of said dressing material, especially also the resting of the wound required for the initial healing process within the first days subsequent to taking care of the wound is guaranteed. The dressing material, according to the subject invention, also combines the advantages of healing the wound in a wet milieu with the advantage of a healing process of a long-period rest of the wound, which assists the healing process, especially within the first days, considerably.

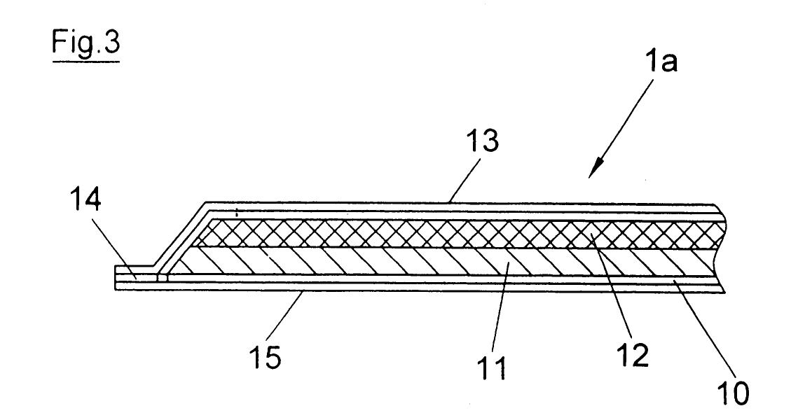

FIG. 3 as a further embodiment of the invention shows a schematic view and in cross section a dressing material 1a which is again made of a plurality of layers, and also serves the improved healing of the wound in a wet milieu. The dressing material 1a comprises a lower layer 10 with which this material is applied onto the wound. This layer 10 which forms the sterile coating on the wound, is made up by a sterile perforated flat material, such as a perforated or grid-like film of suitable plastics material, for example PVC or silicon.

Upon layer 10 a layer 11 is provided, which serves as a distributor layer for the treatment liquid, and is made of a sterile soft and absorbent material, such as gauze or a non-woven material (fleece). On the surface of layer 10 opposite to layer 11 another layer 12 is provided, which includes the liquid treatment medium in capsulated form so that by opening and breaking the layer 12 resp. the capsules there, the treatment medium is distributed over layer 11 to the underside of the distributor material, and thus is discharged to the wound area. Above layer 12 a cover layer 13 is provided which is made of semipermeable flat material, which means a flat material which is not permeable or substantially impermeable for the liquid treatment medium, however, is permeable for air and gas.

As shown in FIG. 3 the cover layer 13 covers the dressing material 1a or alternatively the various layers and sheets of said dressing material also laterally, and is provided with a non-irritant adhesive coating 14 on the edge thereof, which is arranged at the level of the underside of layer 10. With a withdrawable cover layer 15 the dressing material 1a is closed in a sterile manner at that side on which it is applied to a wound. Layer 15 when being used on a wound is removed.

When being used, the dressing material, subsequent to removing the cover or protective layer 15 with the then exposed layer 10, is applied onto the wound and is bonded around the wound by means of an adhesive layer 14. By breaking the layer 12 the capsuled treatment liquid then is released.

Preferably, the dressing material 1a is designed so that when changing the dressing the layer 10 of the dressing is separated from the adjacent layer 11, and the layer 10 is maintained on the wound so that the wound still can rest and thus, the healing process is not influenced by changing the dressing. When changing the dressing the freshly used dressing material then is equivalent to the dressing material la, however, without the layer 10, which means the structure is so that in addition to the protective layer 15 also the layer 10 can be removed from the dressing material 1a, if necessary.

As a treatment medium, a solution is used which, for example, is made of an aqueous solution of zinc and iron, besides distilled water and sulphuric acid, namely 10-100 mg Zn and 6.5-65 mg Fe, for example 15-45 mg Zn and 10-30 mg Fe per litre solution, whereby the share of sulphuric acid is chosen so that a pH-value between 2.5 and 3.5, preferably 2.8, is obtained. With a preferred embodiment a 95-97% sulphuric acid is used. The distilled water is double distilled water according to DAB 10. As raw materials, for example, ZnCl₂ and FeSO₄, about 20-209 mg ZnCl₂ and 32-325 mg FeSO₄ per litre solution are suitable.

In the following, a sample for the composition of the treatment solution is given:

One litre solution comprises

| | |
|---|---|
| Distilled water DAB 10 | 980 g |
| FeSO₄ | 0.097 g |
| ZnCL₂ | 0.063 g |

the remaining is sulphuric acid 95-97% with a share until a pH-value of 2.8 is obtained.

In view of the low pH-value of the treatment medium, the medium does not only result in an acid, but becomes an optimum wound milieu. Based on the acid milieu especially, the metabolism improving the healing process is also stimulated. Furthermore, the treatment medium by means of the acid in combination with the iron constituents also operates microbacteriostatically, which means that a milieu is generated which prevents the growth of germs and bacteria, and acts repulsive on them so that inflammations caused by germs entering the wound and developing there will be effectively prevented.

The forementioned treatment medium (solution) is especially suitable in connection with the dressing material 1 or 1a, for example, used in connection with skin transplantations for taking care or covering transplantations spots, but also for treating the donator (for example bottom) where the transplanted skin has been removed. Experiments have been made on pigs by using the treatment medium described above with a constituent of 30 mg zinc and 20 mg iron per litre solution (in the following 0.003% solution), with a comparable solution which only included 10 mg zinc and about 7 mg iron per litre solution (in the following 0.01% solution) with a comparable solution including 100 mg zinc and 66 mg iron per litre solution (in the following 0.001% solution). By means of a further comparative series of experiments, the healing process has been tested using known processes of taking care of wounds.

The results of said experiments on burns are, as generally acknowledged, also relevant for the effectivity of taking care of chronic wounds, such as decubitus, ulcus cruris wound healing defects in connection with diabetes mellitus, etc.

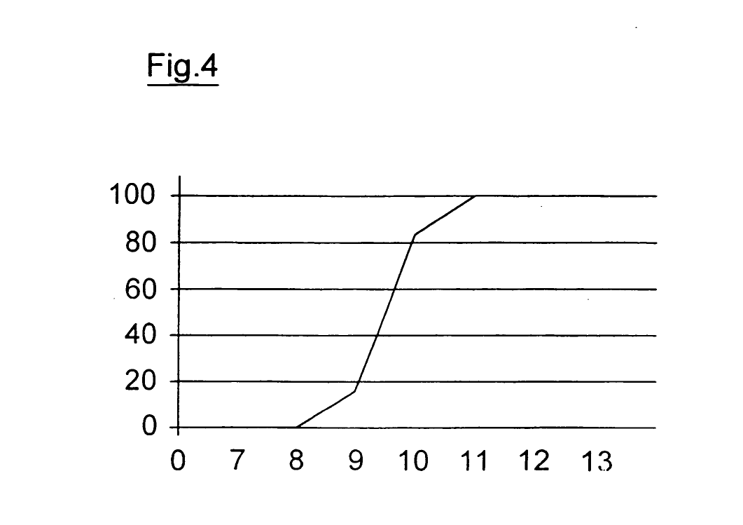

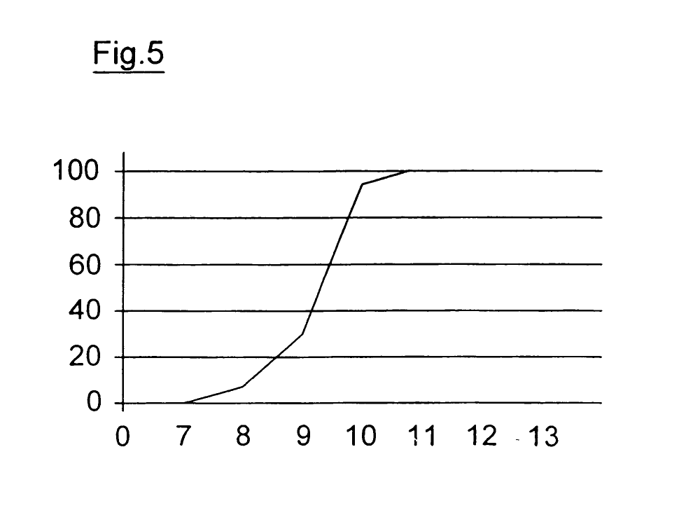

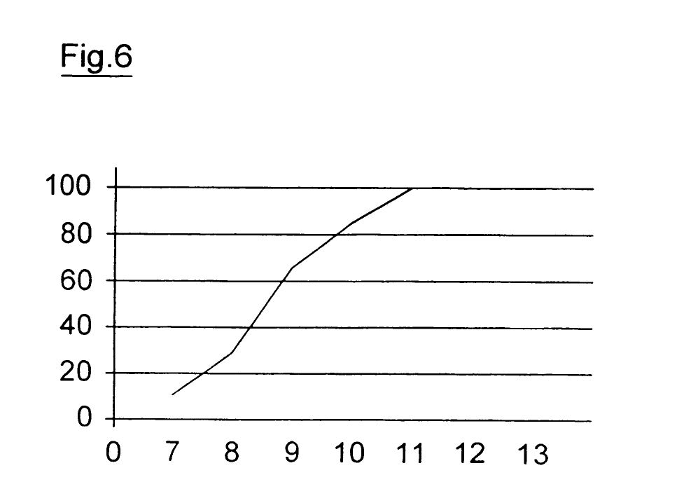

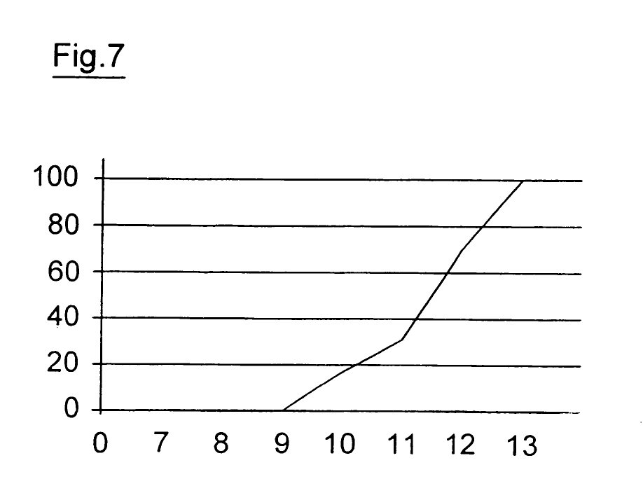

The results are shown in the graphs according to FIGS. 4, 5, 6 and 7, namely in FIG. 4 for the 0.001% solution, in FIG. 5 for the 0.01% solution, in FIG. 6 for the 0.003% solution, and in FIG. 7 for a wound positioned just in open air. The Figures show that the best healing success has been obtained with the 0.003% solution.

Within FIG. 2 reference numeral 16 characterises an indicator which with the shown embodiment comprises a substrate or strips of flat material provided with an indicating material, which in case that the dressing material becomes dry, so that the moisture of said dressing material 1 in use will be reduced below a predetermined value, changes its colour. The indicator 16 is integrated into the dressing material 1 in such a manner that it is visible if the dressing material 1 is applied. With the shown embodiment the indicator 16 is provided underneath that layer which finishes the dressing material at the side opposite to the wound, which means with the dressing material 1 between layer 4 and layer 5, and with the dressing material 1a underneath layer 13.

Suitable materials for indicators which change their colour dependent on the moisture, are known to the expert. Basically, an indicator material is suitable for the subject invention, which dependent on the pH-value changes the colour because if the dressing material runs dry, the pH-value changes.

In addition, or instead of the indicator 16 which indicates if the dressing material runs dry, an indicator can be integrated into the dressing material 1 or 1a, which indicator is based on a possibly increased microbial contamination of the dressing material so that the dressing material can be changed in time. Also this indicator is formed according to indicator 16 and preferably is provided so that it is visible from the dressing applied to the patient, and consists of a carrier material which is provided with a substance altering it s colour with an increased microbial load or contamination, for example also by altering its pH-value.

Basically, there is also the possibility to position a corresponding indicator 16 at that side of the dressing material facing the wound, which indicator indicates the moisture content of the dressing and/or the microbial condition of the dressing.

Above, the invention has been described in connection with embodiments. However, numerous alterations and revisions will be possible without leaving the idea on which the subject invention is based.

1. Dressing material for taking care of wounds, especially for chronic or post-operative wound treatment, using the treatment solution according to any one of the preceeding claims, comprising a plurality of layers, which are arranged adjacent to each other and joined together, namely at least one first perforated layer (2, 10) forming a covering on the wound, a second layer (3, 11), which is preferably multi-layered, is soft and at the same time is absorbent, and a third layer (4, 13) forming the outer surface of the dressing material when being used for treating a wound, characterised in that within the sequence of layers formed by said layers at least one chamber (5, 12) is provided for discharging a liquid treatment medium into the second layer (3, 11).

2. Dressing material according to claim 1, characterised in that at least one chamber (5) comprises at least one connector (6) for supplying a treatment medium at an outer surface of the dressing material (1), and is arranged on a wall for discharging of the treatment medium into the second layer (3) in a finely divided manner.

3. Dressing material according to claim 1 or 2, characterised in that the wall of the at least one chamber (5) is finely perforated.

4. Dressing material according to any one of the preceeding claims, characterised in that the at least one chamber (5) is formed as a channel and with several channel sections and/or windings extends across the surface of the dressing material (1).

5. Dressing material according to claim 4, characterised in that the at least one chamber (5) forms a connector (6) each at two ends thereof.

6. Dressing material according to any one of the preceeding claims, characterised in that the at least one chamber is formed by at least one hose portion (5).

7. Dressing material according to any one of the preceeding claims, characterised in that the at least one chamber (5, 12) is arranged between the third layer (4, 13) forming the outer surface of the dressing material. and a further layer, for example said second layer (3, 11).

8. Dressing material according to any one of the preceeding claims, characterised in that the at least one chamber (5, 12) is provided on that side of the second layer opposite to the first layer and adjacent thereto.

9. Dressing material according to any one of the preceeding claims, characterised in that the at least one chamber (5, 12) is provided in the second layer (3, 11) preferably formed as a multi-layer.

10. Dressing material according to any one of the preceeding claims, characterised in that the third layer (4, 13) is a semi-permeable layer, allowing the entrance of gas and air, however, forms a barrier for liquids.

11. Dressing material according to any one of the preceeding claims, characterised in that at least one connector (6) is extended from one side of the dressing material (1).

12. Dressing material according to any one of the preceeding claims, characterised in that at least one hose portion (5) is fastened to the third layer (4).

13. Dressing material according to any one of the preceeding claims, characterised in that the at least one chamber is formed by at least one capsule for receiving the treatment medium, preferably from a layer (12) of a plurality of capsules.

14. Dressing material according to any one of the preceeding claims, characterised in that the at least one chamber (5, 12) is used for receiving a treatment solution, which per litre solution in addition to water and acid, preferably sulphuric acid, comprises:
10-100 mg zinc (Zn)
6,5-65 mg iron (Fe), for example
15-45 mg zinc
10-30 mg iron,
whereby the share of acid is chosen so that a pH-value of approx. 2.5 until 3.5, preferably of 2.8 is obtained.

15. Dressing material according to any one of the preceeding claims, characterised by at least one indicator (16) for optically indicating the moisture content and/or the microbial load condition of the dressing material.

16. Treatment solution for taking care of wounds, especially for the treatment of chronic and post-operative wounds,
characterised by an aqueous solution which in addition to water and acid, preferably sulphuric acid includes per litre solution:
10-100 mg zinc (Zn)
6.5-65 mg iron (Fe), for example
15-45 mg zinc
10-13 mg iron
whereby the share of acid is chosen so that a pH-value of approximately 2.5 until 3.5, preferably of 2,8 is obtained.

17. Treatment solution according to claim 16, characterised by using 95-97% sulphuric acid.

18. Treatment solution according to claim 16 or 17, characterised by the use of ZnCl₂ as the zinc constituent, preferably in an amount of
10-100 mg per litre solution, for example
46-80 mg per litre solution.

19. Treatment solution according to any one of the preceeding claims, characterised by using FeSO₄ as the iron constituent, preferably in an amount of
32-325 mg per litre solution, for example
80-114 mg per litre solution.

20. Treatment solution according to any one of the preceeding claims, characterised in that the solution per litre solution comprises approx. 30 mg zinc and 20 mg iron, preferably made by using about 63 mg ZnCl₂ and about 97 mg FeSO₄.

21. Treatment solution according to any one of the preceeding claims, characterised in that the aqueous solution in addition to zinc and iron merely includes distillated water and acid.
